(19) United States
(12) Patent Application Publication
(10) Pub. No.: US 2019/0388061 A1
(43) Pub. Date: Dec. 26, 2019

(54) ULTRASOUND DIAGNOSIS APPARATUS DISPLAYING SHEAR WAVE DATA FOR OBJECT AND METHOD FOR OPERATING SAME

(71) Applicant: SAMSUNG MEDISON CO., LTD., Hongcheon-gun (KR)

(72) Inventors: Sun-mo YANG, Hongcheon-gun (KR); Deok-gon KIM, Hongcheon-gun (KR); Seung-ju LEE, Hongcheon-gun (KR); Gil-ju JIN, Hongcheon-gun (KR); Hyoung-ki LEE, Seongnam-si (KR)

(73) Assignee: SAMSUNG MEDISON CO., LTD., Hongcheon-gun (KR)

(21) Appl. No.: 16/480,581

(22) PCT Filed: Apr. 17, 2017
(86) PCT No.: PCT/KR2017/004100

§ 371 Date: Jul. 24, 2019

(30) Foreign Application Priority Data
Jan. 25, 2017 (KR) 10-2017-0012047

(51) Int. Cl. A61B 8/00; A61B 8/08

(52) U.S. Cl. CPC A61B 8/465; A61B 8/085; A61B 8/5207

(57) ABSTRACT

Provided are an ultrasound diagnosis apparatus for displaying shear wave data relating to an object and an operation method thereof. The ultrasound diagnosis apparatus includes a processor configured to obtain the shear wave data of the object with respect to each of a plurality of cross sections spaced apart by a predetermined distance and set a region of interest (ROI) in a reference cross section among the plurality of cross sections; and a display displaying a graphic user interface (GUI) representing shear wave data of a region corresponding to a same location as that of the ROI in the plurality of cross sections along a depth direction.

### TECHNICAL FIELD

The disclosure relates to an ultrasound diagnosis apparatus for displaying shear wave data related to an object and an operation method thereof, and more particularly to, a graphic user interface (GUI) for displaying shear wave data on the same region of a plurality of cross sections in three-dimensional ultrasound volume data.

### BACKGROUND ART

An ultrasound diagnosis apparatus irradiates an ultrasound signal generated by a transducer of a probe to an object and receives information of an echo signal reflected from the object to obtain at least one image of a site inside the object.

Recently, a technology of obtaining a three-dimensional (3D) ultrasound image through a 3D ultrasound probe and diagnosing a lesion has been used. The 3D ultrasound image may be displayed using 3D ultrasound volume data obtained by scanning a plurality of cross sections. A user using the ultrasound diagnosis apparatus must go through an input operation of rotating or moving a cross section to be observed among the plurality of cross sections in the 3D ultrasound volume data by using a user input device such as a knob or a trackball so as to view the cross section including the lesion. Also, a current 3D ultrasound volume data display method has a technical limitation that data values of the remaining cross sections other than a currently displayed cross section may not be displayed simultaneously in case where the user compares data of a specific point of the 3D ultrasound volume data with data of the plurality of cross sections.

### DESCRIPTION OF EMBODIMENTS

### Technical Problem

Provided are an ultrasound diagnosis apparatus that displays a graphic user interface (GUI) representing shear wave data of a plurality of cross sections included in three-dimensional (3D) ultrasound volume data of an object and an operation method thereof.

### Advantageous Effects of Disclosure

An ultrasound diagnosis apparatus according to an embodiment of the disclosure may display a graphic user interface (GUI) representing not only shear wave data with respect to a region of interest (ROI) of a displayed reference cross section image but also shear wave data of a region corresponding to the same location as that of a ROI within a plurality of cross sections on the front and rear of the reference cross section image. Therefore, a user of the ultrasound diagnosis apparatus may simultaneously identify not only the shear wave data of the ROI of the reference cross section image that the user is currently viewing but also the shear wave data along a depth direction of a tissue corresponding to the same location as that of the ROI. Accordingly, it is possible to measure elasticity of the tissue through the shear wave data and to improve diagnosis intuitiveness and user convenience in analyzing lesion information.

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a conceptual diagram for explaining a method, performed by an ultrasound diagnosis apparatus, of displaying a B-mode image of an object, a reference cross section image and a graphic user interface (GUI), according to an embodiment of the disclosure.

FIG. 2 is a block diagram showing a structure of an ultrasound diagnosis apparatus according to an embodiment of the disclosure.

FIG. 3 is a flowchart illustrating a method, performed by an ultrasound diagnosis apparatus, of displaying shear wave data of an object, according to an embodiment of the disclosure.

FIGS. 4A and 4B are diagrams for explaining a process in which an ultrasound diagnosis apparatus generates shear waves in an object, according to an embodiment of the disclosure, wherein FIG. 4B is a diagram for explaining a progress of shear waves.

FIG. 5 is a flowchart illustrating a method, performed by an ultrasound diagnosis apparatus, of obtaining shear wave data of an object using an ultrasound probe, according to an embodiment of the disclosure.

FIGS. 6A through 6E are diagrams illustrating GUIs used by an ultrasound diagnosis apparatus to display shear wave data of a plurality of cross sections, according to an embodiment of the disclosure.

FIG. 7 is a diagram for explaining a method, performed by a display of an ultrasound diagnosis apparatus, of displaying images of a median plane, a sagittal plane, and a horizontal plane of 3D ultrasound volume data and a 3D volume image, according to an embodiment of the disclosure.

FIG. 8 is a diagram for explaining a method, performed by an ultrasound diagnosis apparatus, of displaying GUIs representing shear wave data of a plurality of cross sections on a reference cross section of 3D ultrasound volume data, according to an embodiment of the disclosure.

FIG. 9 is a diagram for explaining a method, performed by an ultrasound diagnosis apparatus, of displaying a reference cross section of 3D ultrasound volume data together with GUIs representing shear wave data of a plurality of cross sections, according to an embodiment of the disclosure.

FIGS. 10A and 10B are diagrams for explaining a method, performed by an ultrasound diagnosis apparatus, of displaying GUIs representing shear wave data of a reference cross section and a plurality of cross sections of 3D ultrasound volume data together, according to an embodiment of the disclosure.

FIGS. 11A and 11B are diagrams for explaining a method, performed by an ultrasound diagnosis apparatus, of displaying thumbnail images of a plurality of cross sections of 3D ultrasound volume data together with GUIs representing shear wave data of the plurality of cross sections on images stored in a memory, according to an embodiment of the disclosure.

FIGS. 12A to 12C are diagrams for explaining a method performed by an ultrasound diagnosis apparatus, of displaying only at least one cross section of interest among a plurality of cross sections of 3D ultrasound volume data, according to an embodiment of the disclosure.

FIG. 13 is a block diagram showing a structure of an ultrasound diagnosis apparatus according to an embodiment of the disclosure.

FIG. 14 is a diagram illustrating ultrasound diagnosis apparatuses according to an embodiment of the disclosure.

### BEST MODE

In accordance with an aspect of the disclosure, an ultrasound diagnosis apparatus for displaying shear wave data relating to an object includes a processor configured to obtain the shear wave data of the object with respect to each of a plurality of cross sections spaced apart by a predetermined distance and set a region of interest (ROI) in a reference cross section among the plurality of cross sections; and a display displaying a graphic user interface (GUI) representing shear wave data of a region corresponding to a same location as that of the ROI in the plurality of cross sections along a depth direction

In accordance with another aspect of the disclosure, a method of displaying shear wave data relating to an object includes obtaining the shear wave data of the object with respect to each of a plurality of cross sections spaced apart by a predetermined distance; setting a ROI in a reference cross section among the plurality of cross sections; and displaying a graphic user interface (GUI) representing shear wave data of a region corresponding to a same location as that of the ROI in the plurality of cross sections along a depth direction.

In accordance with another aspect of the disclosure, a computer-readable recording medium having recorded thereon a program for executing the method in a computer is provided.

### MODE OF DISCLOSURE

This application is based on and claims priority to Korean Patent Application No. 10-2017-0012047, filed on Jan. 25, 2017, in the Korean Intellectual Property Office.

The present specification describes principles of the disclosure and sets forth embodiments thereof to clarify the scope of the disclosure and to allow those of ordinary skill in the art to implement the embodiments of the disclosure. The present embodiments of the disclosure may have different forms and should not be construed as being limited to the descriptions set forth herein.

Like reference numerals refer to like elements throughout. The present specification does not describe all components in the embodiments of the disclosure, and common knowledge in the art or the same descriptions of the embodiments of the disclosure will be omitted below. The term ‘part’ or ‘portion’ used herein may be implemented using hardware or software, and according to embodiments of the disclosure, a plurality of ‘parts’ or ‘portions’ may be formed as a single unit or element, or one ‘part’ or ‘portion’ may include a plurality of units or elements. Hereinafter, the operating principles and embodiments of the disclosure will be described in detail with reference to the accompanying drawings.

In the present specification, an image may include any medical image obtained by various medical imaging apparatuses such as a magnetic resonance imaging (MRI) apparatus, a computed tomography (CT) apparatus, an ultrasound imaging apparatus, or an X-ray apparatus.

Also, in the present specification, an ‘object’, which is a thing to be imaged, may include a human, an animal, or a part thereof. For example, an object may include a part of a human, that is, an organ or a tissue, or a phantom.

Furthermore, in the present specification, a ‘user’ may be, but is not limited to, a medical expert, such as a medical doctor, a nurse, a medical laboratory technologist, or a technician who repairs a medical apparatus.

Throughout the specification, an ‘ultrasound image’ refers to an image of an object processed based on ultrasound signals transmitted to the object and reflected therefrom.

Also, in the present specification, a ‘region of interest (ROI)’ may include not only a region including a predetermined area but also a point corresponding to a specific location on an ultrasound image.

Also, in the present specification, expressions such as “first”, “second”, or “1-1th” are exemplary terms for designating different components, entities, data units, images, pixels or patches. Therefore, the expressions such as “first”, “second”, or “1-1th” do not indicate order between the components or indicate priority.

Reference will now be made in detail to embodiments, examples of which are illustrated in the accompanying drawings. In the following description, well-known functions or constructions are not described in detail so as not to obscure the embodiments with unnecessary detail.

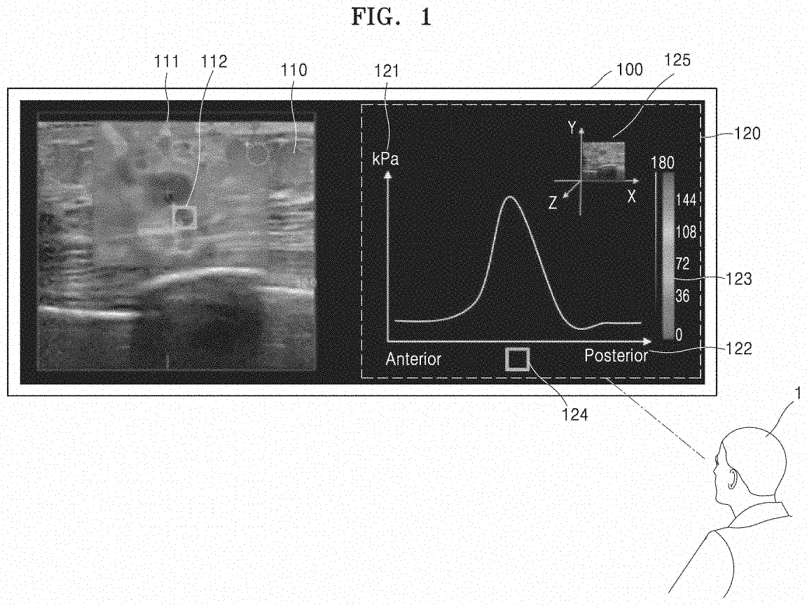

FIG. 1 is a conceptual diagram for explaining a method, performed by an ultrasound diagnosis apparatus 100, of displaying a B-mode image 110 of an object, a reference cross section image 111 and a graphic user interface (GUI) 120 according to an embodiment of the disclosure.

Referring to FIG. 1, the ultrasound diagnosis apparatus 100 may display the reference cross section image 111 of three-dimensional (3D) ultrasound volume data of the object. In an embodiment, the ultrasound diagnosis apparatus 100 may induce a shear wave by irradiating ultrasound waves to the object using an ultrasound probe or the like, and may cause a displacement of a tissue within the object. Thereafter, the ultrasound diagnosis apparatus 100 may obtain the 3D ultrasound volume data by scanning the object through a 3D volume acquisition method or a 3D plane scan method. In an embodiment, the reference cross section image 111 may be a shear wave image including shear wave data of the object. The ultrasound diagnosis apparatus 100 may overlap and display the reference cross section image 111 on the B-mode image 110.

The ultrasound diagnosis apparatus 100 may display tissues in different colors according to a shear wave data value of the reference cross section image 111. For example, the ultrasound diagnosis apparatus 100 may display a point corresponding to a hard tissue and the shear wave data value close to 0 in a blue color and a point corresponding to a relatively soft tissue and the shear wave data value close to 180 in a red color.

The ultrasound diagnosis apparatus 100 may receive a user input for selecting any one of a plurality of cross sections included in the 3D ultrasound volume data and setting the selected one as the reference cross section image 111. The ultrasound diagnosis apparatus 100 may set the reference cross section image 111 based on the user input and output the reference cross section image 111 to a display.

The ultrasound diagnosis apparatus 100 may receive a user input for setting a region of interest (ROI) 112 on the reference cross section image 111. The ultrasound diagnosis apparatus 100 may set the ROI 112 based on the user input and display the ROI 112 on the reference cross section image 111. In FIG. 1, the ROI 112 is indicated in the form of a box, but a method of indicating the ROI 112 is not limited thereto. In an embodiment, the ROI 112 may be displayed differently from other regions on the reference cross section image 111 using a graphical indicator or a marker, including arrows, text, figures, etc.

The ultrasound diagnosis apparatus 100 may display the GUI 120 representing a shear wave data value of a region corresponding to the same location as that of the ROI 112 set in the reference cross section image 111 within the plurality of cross sections included in the 3D ultrasound volume data along a Z-axis depth direction. In an embodiment, the ultrasound diagnosis apparatus 100 may display the GUI 120 along with the reference cross section image 111 of the object, but is not limited thereto.

In the embodiment shown in FIG. 1, the GUI 120 may be a graph that displays distance information 122 of the plurality of cross sections arranged along the Z-axis depth direction on an X-axis and displays the shear wave data value 121 of the region corresponding to the same location as that of the ROI 112 of the plurality of cross sections on a Y-axis. The distance information 122 of the plurality of cross sections may mean relative location information of the plurality of cross sections spaced apart from each other in an anterior or posterior side with respect to a location of the reference cross section image 111. The shear wave data value 121 displayed on the Y-axis may be a shear modulus, and a unit thereof may be kPa, but is not limited thereto. The GUI 120 may include a color bar interface 123 mapped to different colors according to a magnitude of the shear wave data value 121. The color bar interface 123 may display a relationship between a color and a shear wave data value displayed on the tissue on the reference cross section image 111.

In an embodiment, the GUI 120 may include a location indicator 124 and a 3D location coordinate system interface 125 that indicate a relative location of the reference cross section image 111 in which the ROI 112 is set.

The shear wave image is an image technique that induces a shear wave to an object using an ultrasound probe and measures a velocity change according to a shear wave of the tissue in the object to display quantitatively a numerical value. A location, magnitude, and shear wave data value may be different in different cross sections along the Z-axis depth direction even in the same tissue on the 3D ultrasound volume data. In order to analyze shear wave information of a specific tissue, conventional 3D ultrasound volume data must involve an input to rotate 3D volume data using a knob, a trackball, or the like, or select a cross section to be observed among a plurality of cross sections. Shear wave data values in the remaining cross sections other than the currently viewed reference cross section image 111 and a location of the specific tissue are not identified with respect to the specific tissue corresponding to a ROI, which causes inconvenience that makes it impossible to compare the shear wave data values of the remaining cross sections with the shear wave data value of the reference cross section displayed on the reference cross section image 111.

In the embodiment shown in FIG. 1, the ultrasound diagnosis apparatus 100 may display the GUI 120 representing not only the shear wave data with respect to the ROI 112 of the displayed reference cross section image 111 but also shear wave data of the region corresponding to the same location as that of the ROI 112 in the plurality of cross sections disposed in the anterior or posterior side of the reference cross section image 111. Thus, a user 1 may simultaneously identify not only the shear wave data of the ROI 112 of the currently viewed reference cross section image 111 but also the shear wave data along the Z-axis depth direction of a tissue corresponding to the same location as that of the ROI 112. Therefore, it is possible to measure the shear wave of the tissue through the shear wave data and to improve intuitiveness of a diagnosis and user convenience in analyzing lesion information.

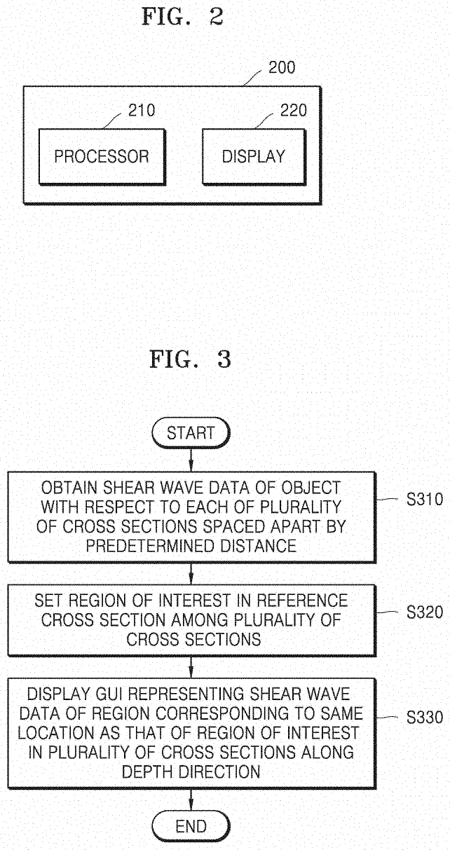

FIG. 2 is a block diagram showing a structure of an ultrasound diagnosis apparatus 200 according to an embodiment of the disclosure. In an embodiment, the ultrasound diagnosis apparatus 200 may be implemented in a portable type as well as a cart type. Examples of a portable ultrasound diagnosis apparatuses include, but are not limited to, a PACS viewer, a smart phone, a laptop computer, a PDA, a tablet PC, and the like.

Referring to FIG. 2, the ultrasound diagnosis apparatus 200 may include a processor 210 and a display 220. However, only indispensable components of the ultrasound diagnosis apparatus 200 are shown in FIG. 2, and the ultrasound diagnosis apparatus 200 may further include other components. In an embodiment, the ultrasound diagnosis apparatus 200 may further include an ultrasound probe for irradiating a focused beam to an object to induce a displacement in a tissue within the object.

The processor 210 may receive an echo signal reflected from the object to which shear wave is induced to obtain 3D ultrasound volume data related to the object. The processor 210 may obtain shear wave data with respect to each of a plurality of cross sections included in the obtained 3D ultrasound volume data.

The plurality of cross sections may be spaced apart by a predetermined distance along a Z-axis depth direction. The distance that the plurality of cross sections are spaced apart may be any value previously set in a memory in the processor 210, but is not limited thereto. In an embodiment, the ultrasound diagnosis apparatus 200 may further include a user input unit. The user input unit may receive a user input for setting the distance that the plurality of cross sections are spaced apart. The number of the plurality of cross sections may be set based on a set distance value but is not limited thereto. The user input unit may receive a user input for setting the number of the plurality of cross sections.

The processor 210 may set a reference cross section among the plurality of cross sections. The processor 210 may set a ROI on the reference cross section and obtain shear wave data of a region corresponding to the same location as that of the ROI in the plurality of cross sections. The shear wave data obtained by the processor 210 may include at least one of a shear modulus, a Young's modulus, and a reliability measurement index.

The processor 210 may include a memory storing at least one of a program, an algorithm, and application data for setting the reference cross section and the ROI and obtaining the shear wave data of the region corresponding to the same location as that of the ROI in the plurality of cross sections, and a hardware unit including a processor for processing the program, the algorithm or the application data stored in the memory. For example, the processor 210 may include a processor including at least one of a central processing unit, a microprocessor, and a graphic processing unit. At this time, the memory and the processor may be configured as a single chip, but are not limited thereto.

The display 220 may display a GUI representing the shear wave data of the plurality of cross sections obtained by the processor 210.

The display 220 may be configured as a physical device including at least one of, for example, a CRT display, an LCD display, a PDP display, an OLED display, an FED display, an LED display, a VFD display, a digital light processing (DLP) display, a flat panel display, a 3D display, and a transparent display but is not limited thereto. In an embodiment, the display 220 may be configured as a touch screen including a touch interface. When the display 220 is configured as the touch screen, the display 220 may be a component integrated with the user input unit.

The display 220 may display the GUI representing the shear wave data of the region corresponding to the same location as that of the ROI set in the reference cross section along the Z-axis depth direction with respect to the plurality of cross sections. In an embodiment, the display 220 display the GUI representing the shear wave data of the region corresponding to the same location as that of the ROI of each of a first cross section that is spaced apart from the reference cross section in a first direction along the Z-axis depth direction and a second cross section that is spaced from the reference cross section in a second direction opposite to the first direction.

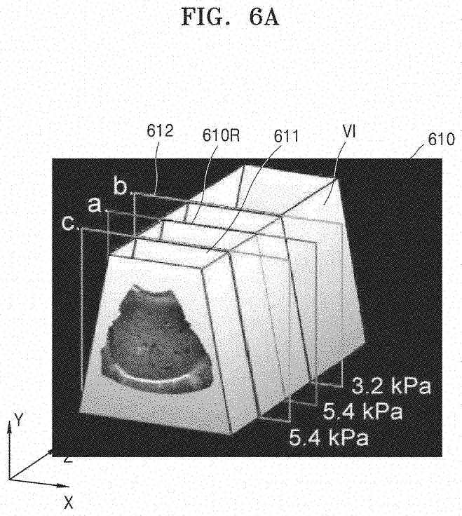

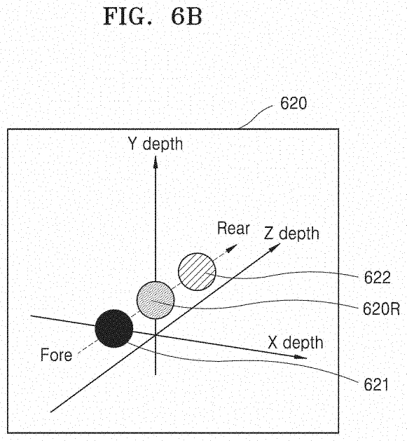

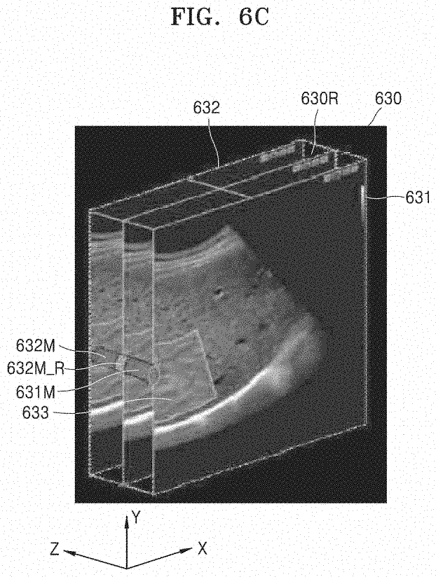

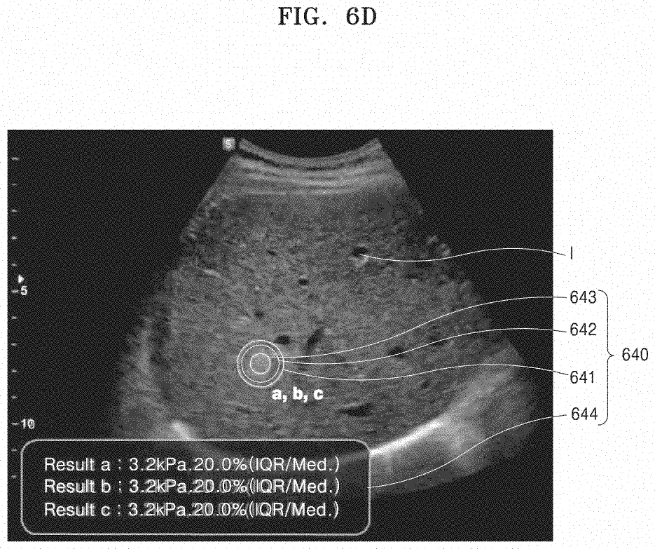

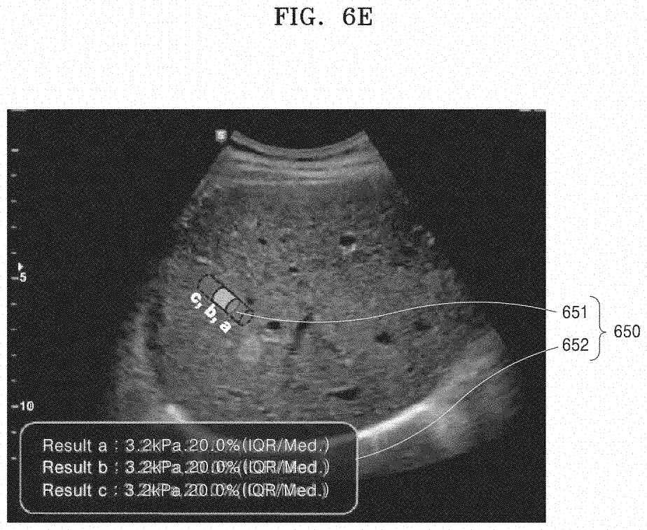

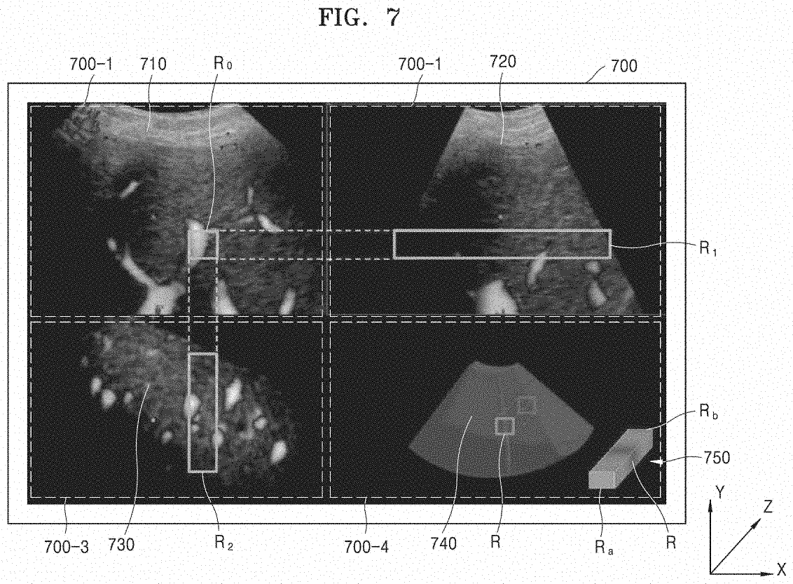

The display 220 may display the shear wave data of the plurality of cross sections in the GUI including at least one of a 3D marker, text, a dotted line, and a 3D coordinate value. However, the GUI displayed by the display 220 is not limited to the above listed examples. A detailed description of the GUI will be provided later in FIGS. 6A to 6E.

In an embodiment, the display 220 may overlap and display the GUI on a location corresponding to the ROI on a reference cross section image. In another embodiment, the display 220 may display the GUI together with a shear wave image of the reference cross section on a location spaced apart by a predetermined distance. Also, in another embodiment, the display 220 may display the reference cross section image on a first region on a display screen and display the GUI on a second region on the display screen.

FIG. 3 is a flowchart illustrating a method, performed by an ultrasound diagnosis apparatus, of displaying shear wave data of an object according to an embodiment of the disclosure.

In operation S310, the ultrasound diagnosis apparatus obtains the shear wave data of the object with respect to each of a plurality of cross sections spaced by a predetermined distance. In an embodiment, the ultrasound diagnosis apparatus may induce a shear wave to the object using an ultrasound probe and cause a displacement of a tissue within the object and then scan the object through a 3D volume acquisition method or a 3D plane scan method to obtain 3D ultrasound volume data. The ultrasound diagnosis apparatus may obtain the shear wave data of the plurality of cross sections included in the 3D ultrasound volume data. The plurality of cross sections may be spaced apart by the predetermined distance in a Z-axis depth direction.

In operation S320, the ultrasound diagnosis apparatus sets a ROI in a reference cross section among the plurality of cross sections. In an embodiment, the ultrasound diagnosis apparatus may include a user input unit for receiving a user input. The user input unit may include, but is not limited to, a hardware component such as a key pad, a mouse, a trackball, a touch pad, a touch screen, a jog switch, etc. In an embodiment, the user input may receive a user input for setting the ROI in a shear wave image of the reference cross section among the plurality of cross sections. The ultrasound diagnosis apparatus may set the ROI on a reference cross section image based on the received user input.

In operation S330, the ultrasound diagnosis apparatus displays a GUI representing shear wave data of a region corresponding to the same location as that of the ROI in the plurality of cross sections along the depth direction. In an embodiment, the GUI may display shear wave data of the region corresponding to the same location as that of the ROI with respect to a cross section disposed in an anterior or posterior side of the reference cross section image along the X-axis depth direction.

In an embodiment, the ultrasound diagnosis apparatus may display the shear wave image of the reference cross section and may display the GUI on a location corresponding to the ROI of the shear wave image of the reference cross section in an overlapping manner. However, the disclosure is not limited thereto, and the ultrasound diagnosis apparatus may display the shear wave image of the reference cross section along with the GUI on a location spaced apart from the GUI.

In an embodiment, the ultrasound diagnosis apparatus may display a location indicator and a 3D location coordinate system interface, which represent a relative location of the reference cross section image in which the ROI is set among the plurality of cross sections.

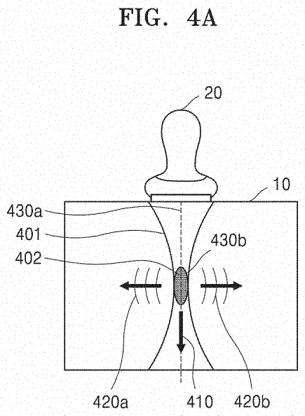

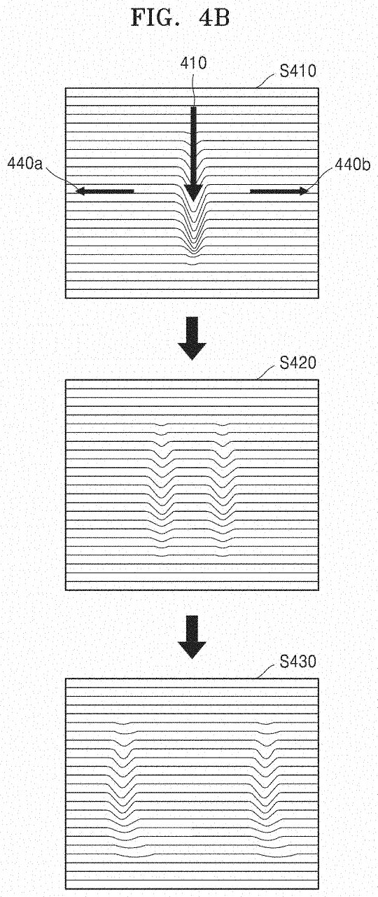

FIGS. 4A and 4B are diagrams for explaining a process in which an ultrasound diagnosis apparatus generates shear waves 420a and 420b in an object 10 according to an embodiment of the disclosure. FIG. 4B is a diagram for explaining a progress of shear waves.

Referring to FIG. 4A, an ultrasound probe 20 may irradiate a focus beam 401 to the object 10 to induce a displacement 410 of the object 10. When the object 10 is irradiated with the focus beam 401, the displacement 410 of the object 10 is induced in a focusing location 402 where the focus beam 401 is focused. The displacement 410 of the object 10 causes the shear waves 420a and 420b proceeding in a orthogonal direction with respect to the displacement 410 from a point where the displacement 410 occurs. The shear waves 420a and 420b generated in the focusing location 402 proceed along the orthogonal direction with respect to the displacement 410 and gradually attenuate and disappear. A mode for capturing the shear waves 420a and 420b of the object 10 is referred to as a shear wave mode. The shear wave mode may include a 2D shear wave measurement mode and a point shear wave measurement mode but is not limited thereto.

In the present specification, the ultrasound diagnosis apparatus is described as obtaining shear wave data of the object 10 using the point shear wave measurement mode. However, a method of obtaining the shear wave data is not limited thereto. The shear wave data may be obtained in the 2D shear wave measurement mode.

In the embodiment shown in FIG. 4A, the ultrasound diagnosis apparatus may generate shear waves in an object by irradiating a focus beam to a focusing location 430b on a determined focus beam irradiation line 430a.

FIG. 4B is a diagram for explaining a progress of shear waves. In an embodiment, the shear waves generated by the ultrasound probe 20 may induce the displacement 410 at a focusing location and may proceed in directions of 440a and 440b as shown in S410 to S430.

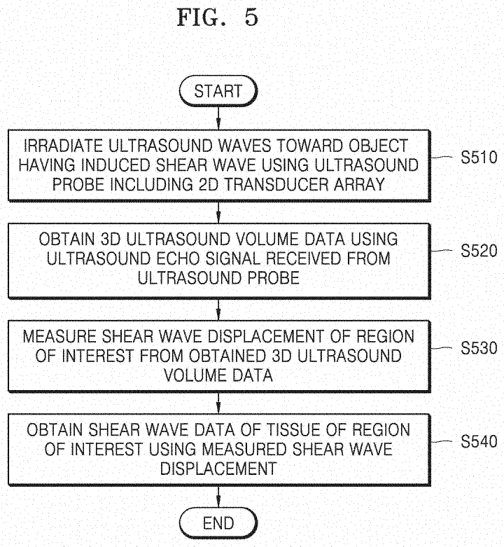

FIG. 5 is a flowchart illustrating a method, performed by an ultrasound diagnosis apparatus, of obtaining shear wave data of an object using an ultrasound probe according to an embodiment of the disclosure.

In operation S510, the ultrasound diagnosis apparatus irradiates ultrasound waves toward an object using the ultrasound probe including a 2D transducer array. In an embodiment, an acoustic radiation force impulse (ARFI), such as a diagnosis ultrasound wave, may be applied to an inside of a body to quantitatively analyze a shear wave for each tissue within the object to cause a displacement of a tissue. As such, shear waves are induced in the tissue within the object by the ARFI, and thus the displacement of the tissue may occur.

In an embodiment, the ultrasound probe may include the 2D transducer array. The ultrasound probe includes the 2D transducer array, and thus 3D ultrasound volume data may be obtained at a high speed.

In operation S520, the ultrasound diagnosis apparatus obtains the 3D ultrasound volume data by applying the ultrasound echo signal received from the ultrasound probe. In an embodiment, the ultrasound probe may irradiate ultrasound waves through a 3D volume acquisition method by which a 3D volume of the object may be scanned at a time using the 2D transducer array. In another embodiment, the ultrasound probe may irradiate ultrasound waves through a 3D plane scan method by which the object may be scanned in a plane unit to generate the 3D ultrasound volume data of the object using the 2D transducer array.

The ultrasound diagnosis apparatus may receive the ultrasound echo signal reflected from the object and obtain the 3D ultrasound volume data. In an embodiment, the ultrasound diagnosis apparatus may image process the received echo signal to obtain 3D ultrasound images of thousands of frames per second. That is, the ultrasound diagnosis apparatus may obtain the 3D ultrasound images of thousands of frames by beam-forming and processing the echo signal received from the ultrasound probe. A method of processing an ultrasound image by using an echo signal is obvious to one of ordinary skill in the art, and thus a detailed description thereof is omitted.

In operation S530, the ultrasound diagnosis apparatus measures a shear wave displacement of a ROI from the obtained 3D ultrasound volume data. In an embodiment, the ultrasound diagnosis apparatus may measure the shear wave displacement from the 3D ultrasound images obtained from the 3D ultrasound volume data. The shear wave displacement may mean measuring of a 3D movement of the shear wave. That is, the measured shear wave displacement may have displacement components corresponding to X, Y, and Z axes of any 3D coordinate space. A method of measuring the shear wave displacement by analyzing the shear wave movement represented by the 3D ultrasound images is obvious to one of ordinary skill in the art, and thus a detailed description thereof is omitted.

In operation S540, the ultrasound diagnosis apparatus obtains shear wave data of a tissue of the ROI using the measured shear wave displacement. In an embodiment, the ultrasound diagnosis apparatus may set the ROI on any one of a plurality of cross sections of the 3D ultrasound volume data and obtain shear wave data of a region corresponding to the same location as that of the set ROI among the plurality of cross sections. For example, the ultrasound diagnosis apparatus may set a region including a specific tissue in the object as the ROI, and measure a value of the shear wave data of the specific tissue in a Z-axis depth direction.

FIGS. 6A through 6E are diagrams illustrating GUIs 610, 620, 630, 640 and 650 used by an ultrasound diagnosis apparatus to display shear wave data of a plurality of cross sections according to an embodiment of the disclosure.

Referring to FIG. 6A, the first GUI 610 may include a 3D volume image VI and a plurality of cross section images 611 and 612 including a reference cross section image 610R. The 3D volume image VI may be a graphic representation of 3D ultrasound volume data of an object.

The reference cross section image 61OR may be disposed between the first cross section image 611 and the second cross section image 612. The reference cross section image 610R, the first cross section image 611, and the second cross section image 612 may be spaced apart by a predetermined distance in a Z-axis depth direction on the 3D volume image VI. The first cross section image 611 is shown on the front of the reference cross section 610R, which may mean that a first cross section is a front side compared to a reference cross section. Likewise, the second cross section image 612 is shown on the rear of the reference cross section 610R, which may mean that a second cross section is a rear side compared to the reference cross section.

Cross section image identification characters a, b, and c may be respectively displayed on the reference cross section image 610R, the first cross section image 611, and the second cross section image 612. Shear modulus values 5.4 kPa and 3.2 kPa measured in the respective cross sections may be displayed. For example, the identification character c may be displayed on the reference cross section image 610R, and the shear modulus value of the reference cross section may be displayed as 5.4 kPa. The identification character b may be displayed on the first cross section image 611, and the shear modulus value may be 3.2 kPa. Likewise, the identification character b may be displayed on the second cross section image 612, and the shear modulus value may be 3.2 kPa.

The first GUI 610 may display the first cross section image 611 and the second cross section image 612 disposed on the front and rear of the reference cross section image 610R, and display the identification characters thereof and shear wave data, and thus a user may easily compare a location of each cross section and the shear wave data of each cross section in 3D ultrasound volume data.

Referring to FIG. 6B, the second GUI 620 may be a 3D location coordinate system that represents relative locations of a reference cross section, a first cross section, and a second cross section.

The second GUI 620 may set a location of the reference cross section in the 3D location coordinate system to a value of 0 with respect to X-axis and Z-axis, and may include images of the first cross section spaced in a first direction along the Z-axis depth direction with respect to the reference cross section and the second cross section spaced in a second direction along the Z-axis depth direction. In an embodiment, the first direction may be a fore relative to the reference cross section, and the second direction may be a rear relative to the reference cross section.

The second GUI 620 may display a ROI image 620R of the reference cross section, a first ROI image 621, and a second ROI image 622 on the 3D location coordinate system. The first ROI image 621 may be an image graphically showing a region located in the same region as the ROI set in the reference cross section of the first cross section. Likewise, the second ROI image 622 may be an image graphically showing a region located in the same region as the ROI set in the reference cross section of the second cross section.

In FIG. 6B, the ROI image 620R, the first ROI image 621, and the second ROI image 622 are shown as sphere-shaped images, but this is for ease of illustration and are not limited thereto.

The first ROI image 621 is shown on the front of the ROI image 620R, which may mean that the first cross section is a front side compared to the reference cross section. Likewise, the second ROI image 622 is shown on the rear of the ROI image 620R, which may mean that the second cross section is a rear side compared to the reference cross section.

In an embodiment, the ROI image 620R, the first ROI image 621, and the second ROI image 622 may be displayed in different colors. The second GUI 620 may be displayed mainly with a shear wave image or a B-mode image of the reference cross section, which may allow the user to easily compare shear wave data of the ROI of the reference cross section with shear wave data of the first cross section and the second cross section.

Referring to FIG. 6C, the third GUI 630 may include a plurality of cross sections 630, 631, and 632, a shear wave image 633, and 3D markers 630M_R, 631M, and 632M.

A reference cross section image 630R may be disposed between the first cross section image 631 and the second cross section image 632. The reference cross section image 630R, the first cross section image 631, and the second cross section image 632 may be spaced apart by a predetermined distance in the Z-axis depth direction. The first cross section image 631 is shown on the front of the reference cross section image 630R, which may mean that the first cross section is a front side compared to the reference cross section. Likewise, the second cross section image 632 is shown on the rear of the reference cross section image 630R, which may mean that the second cross section is a back side compared to the reference cross section.

The shear wave image 633 may be overlapped and displayed on a region including a ROI of each of the reference cross section image 630R, the first cross section image 631, and the second cross section image 632.

The 3D markers 630M_R, 631 M, and 632M may be overlapped and displayed on the ROI over the reference cross section image 630R, the first cross section image 631, and the second cross section image 632. The marker 630M_R may be displayed on the reference cross section image 630R in a yellow color. The markers 631M and 632M may be displayed in a red color on the first cross section image 631 and the second cross section image 632. This may mean that a value of the shear wave data of a tissue at the location corresponding to the ROI is large in the first cross section and the third cross section and is relatively small in the reference cross section. That is, the shear wave of the tissue in the ROI may be different according to the Z-axis depth direction.

The third GUI 630 may display the 3D markers 630M_R, 631 M, and 632M on the ROI of the plurality of cross section images 630R, 631, and 632 and the same location as that of the ROI, and display colors of the 3D markers 630M_R, 631 M, and 632M differently according to the cross sections, and thus the user may intuitively identify magnitude of shear wave data of a specific tissue along the Z-axis depth direction.

Referring to FIG. 6D, the fourth GUI 640 may include circular markers 641, 642, 643 and a shear wave data indicator 644 displayed on a shear wave image I of the reference cross section. The circular markers 641, 642 and 643 may be displayed on a ROI set on the shear wave image I of the reference cross section.

The first circular marker 641, the second circular marker 642, and the third circular marker 643 may have different sizes of circles and may be displayed in different colors. In an embodiment, the first circular marker 641 may be displayed in a blue color and may be displayed larger than the second circular marker 642 and the third circular marker 643. The second circular marker 642 may be displayed in a green color and may be displayed larger than the third circular marker 643. The third circular marker 643 may be displayed in a yellow color.

The shear wave data indicator 644 may display a value of shear wave data of each of the first circular marker 641, the second circular marker 642, and the third circular marker 643. In an embodiment, values of shear modulus of tissues corresponding to the first circular marker 641, the second circular marker 642, and the third circular marker 643 may all be 3.2 kPa.

Even in case of the same tissue in the ROI in the fourth GUI 640, a cross section located in the foremost side with respect to a user may be displayed as the first circular marker 641 and a cross section located in the rearmost side may be displayed as the third circular marker 643. This is to allow the user to intuitively confirm a value of shear wave data of the cross section located on the foremost side through the first circular marker 641 displayed as the largest circle. Likewise, the user may confirm a value of shear wave data of the cross section located at the rearmost side through the third circular marker 643 displayed as a circle of the smallest size.

Referring to FIG. 6E, the fifth GUI 650 may include a 3D marker 651 and a shear wave data indicator 652.

The 3D marker 651 may be overlapped and displayed on a location of a ROI set on a shear wave image of a reference cross section b. The 3D markers 651 may be displayed in a yellow color on a region corresponding to the reference cross section b and may be displayed in a red color on a first cross section a disposed on the front of the reference cross section b and a second cross section c disposed on the rear of the reference cross section b. This may mean that a value of shear wave data of a tissue at a location corresponding to the ROI is large in the first cross section a and the third cross section c and is relatively small in the reference cross section b. That is, shear wave of the tissue in the ROI may be different according to the Z-axis depth direction.

The fifth GUI 650 may display the 3D marker 651 on the shear wave image of the reference cross section b and display a color of the 3D marker 651 differently on the ROI on the reference cross section b, the first cross section a, and the second cross section b and the same location as that of the ROI, And the color of the 3D marker 651 at the same location are displayed differently, thereby allowing the user to intuitively identify magnitude of shear wave data of a specific tissue along the Z-axis depth direction.

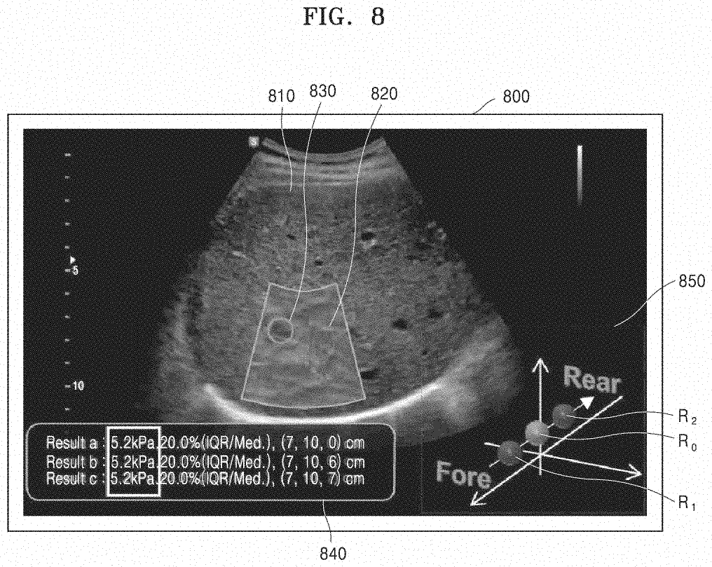

FIG. 7 is a diagram for explaining a method, performed by a display 700 of an ultrasound diagnosis apparatus, of displaying 3D volume images 710, 720, and 730 of a median plane, a sagittal plane, and a horizontal plane and a 3D volume image 740 according to an embodiment of the disclosure. The ultrasound diagnosis apparatus 700 may further display a ROI interface 750.

Referring to FIG. 7, the display 700 may display a median plane ultrasound image 710 of a reference cross section on a first region 700-1 of a display screen, a sagittal plane ultrasound image 720 on a second region 700-2, and a horizontal plane ultrasound image 730 on a third region 700-3. However, the disclosure is not limited thereto, and the display may further display a coronal ultrasound image. The ultrasound diagnosis apparatus 700 may display a 3D volume image 740 and a ROI interface 750 on a fourth region 700-4 of the display screen.

ROI markers R₀, R₁, and R₂ indicating a ROI may be displayed on the median plane ultrasound image 710, the sagittal plane ultrasound image 720, and the horizontal plane ultrasound image 730.

The ROI marker R₀ displayed on the median plane ultrasound image 710 may be a ROI set in a median plane image of a reference cross section currently displayed on the display 700. In an embodiment, the ROI may be set based on a received user input.

The sagittal plane ultrasound image 720 may be a sagittal view image of the reference cross section. A region displayed on the ROI marker R₁ may be viewed on a sagittal plane of the ROI set in the median plane ultrasound image R. That is, the region displayed on the ROI marker R₁ may be a view of the ROI in a Z-axis depth direction.

The horizontal plane ultrasound image 730 may be a horizontal view image of the reference cross section. A region displayed on the ROI marker R₂ may be viewed on a horizontal plane of the ROI set in the median plane ultrasound image R. That is, the region displayed on the ROI marker R₂ may be a view of the ROI in the Z-axis depth direction.

The 3D volume image 740 representing 3D ultrasound volume data of an object in a virtual graphic may be displayed on the fourth region 700-4 of the display 700. A location of the ROI R set in the median plane ultrasound image 710 may be displayed on the 3D volume image 740. The ROI R may be displayed on the 3D volume image 740 at the location corresponding to the ROI in an overlapping manner.

The ROI R may be displayed in a solid line of a rectangular shape having a predetermined region on X-axis and Y-axis on the 3D volume image, and may be displayed in a dotted line in a Z-axis depth direction. A region indicated in the solid line in the ROI R may be the same as a region indicated by the ROI marker R₀ displayed as the ROI in the median plane ultrasound image 710. A region indicated in the dotted line may be the same as regions indicated in the ROI markers R₁ and R₂ displayed on the sagittal plane ultrasound image 720 and the horizontal plane ultrasound image 730 respectively.

The ROI interface 750 may be displayed on the fourth region 700-4 of the display 700. The ROI interface 750 may be the same as a shape of the ROI R displayed on the 3D volume image 740. That is, the ROI interface 750 may be displayed in a square pillar shape having a predetermined region in X-axis and Y-axis directions and having a predetermined length in the Z-axis depth direction. The ROI interface 750 may display values of shear wave data in different colors on the ROI R set in the reference cross section of the 3D ultrasound volume data represented by the 3D volume image 740 and ROI Rₐ and ROI R_{b} of each of a plurality of cross sections corresponding to the same location as that of the ROI R. In an embodiment, a region displayed in a red color on the ROI interface 750 may be the ROI R set in the reference cross section. Also, in the ROI interface 750, a region displayed in a blue color may be a cross section disposed on the front of the reference cross section, that is, the ROI Rₐ in a front side, or a cross section disposed on the rear of the reference cross section, that is, the ROI R_{b} in a rear side.

In the embodiment shown in FIG. 7, the display 700 of the ultrasound diagnosis apparatus may display the median plane ultrasound image 710, the sagittal plane ultrasound image 720, and the horizontal plane ultrasound image 730 including the ROI markers R₀, R₁, and R₂ together and may display the 3D volume image 740 and the ROI interface 750, thereby allowing the user to easily and intuitively identify a location of a ROI and a 3D stereoscopic shape. Also, the ROI interface 750 may display the set ROI in different colors according to values of shear wave data in a plurality of cross sections, thereby allowing the user to easily confirm a difference in the values of the shear wave data of an object along the Z-axis depth direction, and thus user convenience may be improved.

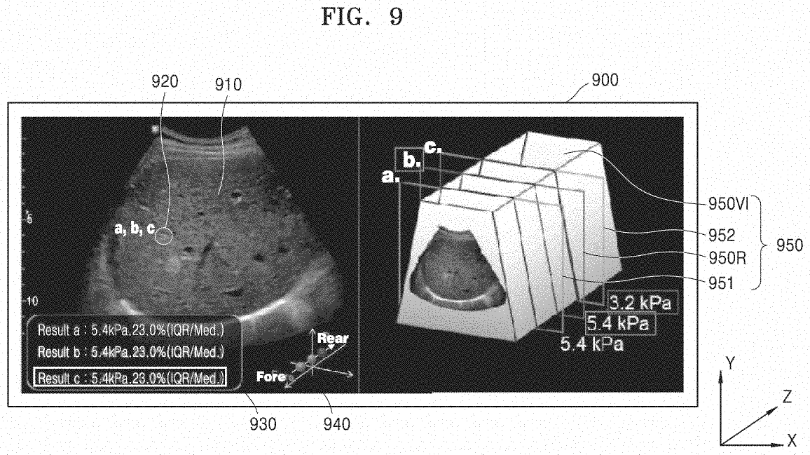

FIG. 8 is a diagram for explaining a method, performed by an ultrasound diagnosis apparatus, of displaying GUIs 830, 840, and 850 representing shear wave data of a plurality of cross sections on a reference cross section image 810 of 3D ultrasound volume data according to an embodiment of the disclosure.

Referring to FIG. 8, a display 800 of the ultrasound diagnosis apparatus may display a B-mode image 810 and a shear wave image 820 of a reference cross section of the 3D ultrasound volume data together with the first GUI 830, the second GUI 840, and the third GUI 850.

The first GUI 830 may be an interface displaying a ROI set on the shear wave image 820 of the reference cross section. In an embodiment, the ROI may be set based on a user input. The first GUI 830 may be overlapped and displayed at a location corresponding to the ROI set on the shear wave image 820.

The second GUI 840 may display a shear wave data value in the ROI of each of a plurality of cross sections including the reference cross section and a 3D location coordinate value of each of the plurality of cross sections.

The third GUI 850 may display a 3D location coordinate system that represents a relative location of the ROI in the plurality of cross sections including the reference cross section. The third GUI 850 may set a location of the ROI of the reference cross section in the 3D location coordinate system to a value of 0 with respect to X-axis and Z-axis, and may include ROI images of each of a first cross section spaced in a first direction along the Z-axis depth direction with respect to the reference cross section and a second cross section spaced in a second direction along the Z-axis depth direction with respect to the reference cross section. The third GUI 850 may display the ROI R₀, the first ROI R₁, and the second ROI R₂ in the 3D location coordinate system. The third GUI 850 is the same as the second GUI 620 illustrated in FIG. 6B, and thus a redundant description thereof is omitted.

In the embodiment shown in FIG. 8, the ultrasound diagnosis apparatus may display the shear wave image 820 of the reference cross section among the plurality of cross sections of the 3D ultrasound volume data of an object together with the GUIs 830, 840 and 850, and thus a user may confirm not only shear wave data of the ROI set on the shear wave image 820 of the reference cross section but also shear wave data values of the front and rear sides of the reference cross section at a time, thereby improving user convenience.

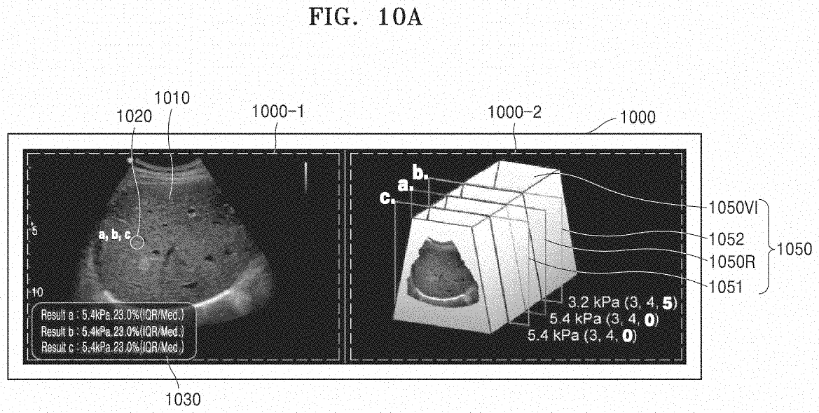

FIG. 9 is a diagram for explaining a method, performed by an ultrasound diagnosis apparatus, of displaying a reference cross section image 910 of 3D ultrasound volume data together with GUIs 930, 940, and 950 representing shear wave data of a plurality of cross sections according to an embodiment of the disclosure.

Referring to FIG. 9, a display 900 of the ultrasound diagnosis apparatus may display a B-mode image 910 of a reference cross section among the plurality of cross sections in the 3D ultrasound volume data, a marker 920 representing a ROI set on the B-mode image 910 of the reference cross section, and the GUIs 930, 940, and 950.

The marker 920 may be displayed at a location corresponding to the set ROI on the B-mode image 910 of the reference cross section in an overlapping manner. The identification characters a, b, and c displayed on the marker 920 may be identification characters with respect to the plurality of cross sections. For example, the reference cross section may be displayed as the character b, a cross section disposed on the front of the reference cross section may be displayed as the character a, and a cross section disposed on the rear of the reference cross section may be displayed as the character b .

The first GUI 930 may display a shear wave data value in the ROI of each of the plurality of cross sections including the reference cross section b. In the embodiment shown in FIG. 9, a shear modulus of the ROI in the reference cross section b may be 5.4 kPa. A shear modulus of a first cross section a disposed on the front side of the reference cross section b may be 5.4 kPa. A shear modulus of a second cross section c disposed on the rear side the reference cross section b may be 3.2 kPa.

The second GUI 940 may display a 3D location coordinate system representing relative locations of the plurality of cross sections a and c including the reference cross section b. The second GUI 940 may set a location of the reference cross section in the 3D location coordinate system to a value of 0 on X-axis and Z-axis, and may include images of the first cross section a spaced in a first direction along the Z-axis depth direction with respect to the reference cross section b and the second cross section c spaced in a second direction along the Z-axis depth direction. The second GUI 940 is the same as the second GUI 620 illustrated in FIG. 6B, and thus a redundant description thereof is omitted.

The third GUI 950 may include a plurality of cross section images 951 and 952 including a 3D ultrasound volume image 950VI and a reference cross section image 950R. The 3D ultrasound volume image 950VI may be a graphical representation of 3D ultrasound volume data of an object.

The reference cross section image 950R may be disposed between the first cross section image 951 and the second cross section image 952. The reference cross section image 950R, the first cross section image 951 and the second cross section image 952 may be spaced apart by a predetermined distance along the Z-axis depth direction on the 3D ultrasound volume image 950VI. The first cross section image 951 is shown on the front of the reference cross section image 950R, which may mean that the first cross section is a front side compared to the reference cross section. Likewise, the second cross section image 952 is shown on the rear of the reference cross section image 950R, which may mean that the second cross section is a back side compared to the reference cross section.

In the embodiment shown in FIG. 9, the cross section image identification characters a, b, and c may be respectively displayed on the reference cross section image 950R, the first cross section image 951, and the second cross section image 952. Shear modulus values 5.4 kPa and 3.2 kPa measured in the respective cross sections may be displayed. For example, the identification character b may be displayed on the reference cross section image 950R, and the shear modulus value may be displayed as 5.4 kPa. The identification character a may be displayed on the first cross section image 951, and the shear modulus value may be 5.4 kPa. The identification character c may be displayed on the second cross section image 952, and the shear modulus value may be 3.2 kPa.

The ultrasound diagnosis apparatus according to the embodiment shown in FIG. 9 may display the B-mode image 910 of the reference cross section together with the third GUI 950 representing relative locations of the cross section images 950R, 951, and 952 of the reference cross section b and the plurality of cross sections a and c, respectively, along the Z-axis depth direction, which allowing a user to easily identify the relative locations of the reference cross section as well as other cross sections. Also, the ultrasound diagnosis apparatus may display the first GUI 930 displaying the shear modulus values of the reference cross section b and the plurality of cross sections a and c together with the second GUI 940 displaying the 3D location coordinate system, thereby improving user convenience.

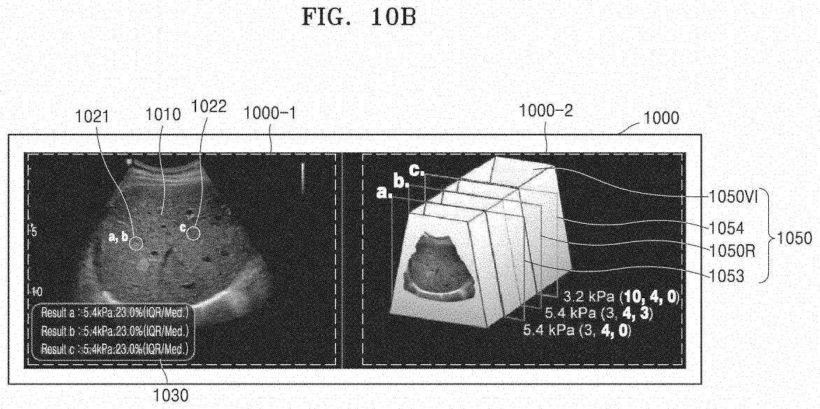

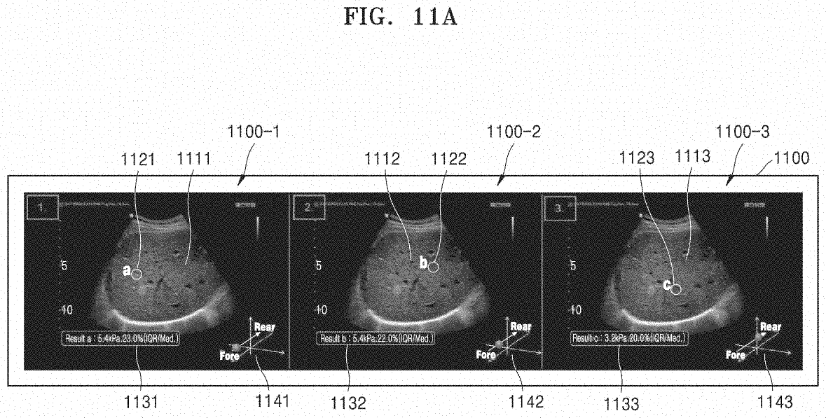

FIGS. 10A and 10B are diagrams for explaining a method, performed by an ultrasound diagnosis apparatus, of displaying GUIs representing shear wave data of a reference cross section and a plurality of cross sections of 3D ultrasound volume data together according to an embodiment of the disclosure.

Referring to FIG. 10A, a display 1000 of the ultrasound diagnosis apparatus may display, on a first region 1000-1, a B-mode image 1010 of the reference cross section, a marker 1020 indicating a ROI, and a first GUI 1030 that displays shear wave data values of the plurality of cross sections. The display 1000 may also display, on a second region 1000-2, a third GUI 1050 including an ultrasound volume image 1050VI, a reference cross section image 1050R and a plurality of cross section images 1051 and 1052. The first GUI 1030 and the third GUI 1050 shown in FIG. 10A are the same as the first GUI 930 and the third GUI 950 shown in FIG. 9, respectively, and thus redundant descriptions thereof are omitted.

On the third GUI 1050, the reference cross section image 1050R may be displayed in the identification character a, and the shear modulus may be displayed as 5.4 kPa. Similarly, the first cross section image 1051 may be displayed in the identification character c, the shear modulus may be displayed as 5.4 kPa, the second cross section image 1052 may be displayed in the identification character b, and the shear modulus may be displayed as 3.2 kPa.

The third GUI 1050 may display a 3D location coordinate value of the ROI indicated by the marker 1020 on the B-mode image 1010 of the reference cross section. For example, the 3D location coordinate value of the ROI in the reference cross section image 1050R may be (3, 4, 0). Similarly, a 3D location coordinate value of the ROI in the first cross section image 1051 may be (3, 4, 0) and a 3D location coordinate value of the ROI in the second cross section image 1052 may be (3, 4, 5). In the embodiment of FIG. 10A, the 3D location coordinate value of the ROI in the first cross section image 1051 is equal to the 3D location coordinate value of the ROI set in the reference cross section image 1050R. This is because shear wave data is obtained by capturing the same region a plurality of times in order to increase accuracy in measuring the shear wave data. The 3D location coordinate value of the ROI of the second cross section image 1052 is the same as the 3D location coordinate value of the ROI of the reference cross section image 1050R in X and Y axis values of 3 and 4, respectively, whereas a Z axis value of 5 is different from a value of 0 of the reference cross section. This may mean that a location of a second cross section is on the rear side of the reference cross section, and a shape and size of a tissue included in the ROI set in a body are kept constant along a Z-axis depth direction.

Referring to FIG. 10B, the display 1000 of the ultrasound diagnosis apparatus may display, on the first region 1000-1, the B-mode image 1010 of the reference cross section, a first marker 1021 indicating the ROI, a second marker 1022, and the first GUI 1030 that displays the shear wave data values of the plurality of cross sections. The display 1000 may also display, on the second region 1000-2, the third GUI 1050 including the ultrasound volume image 1050VI, the reference cross section image 1050R and a plurality of cross section images 1053 and 1054.

Referring to the third GUI 1050, a 3D location coordinate value of the ROI displayed on the reference cross section image 1050R may be (3, 4, 3), and a 3D location coordinate value of the ROI of the third cross section image 1053 that is a cross section disposed on the front side of the reference cross section image 1050R may be (3, 4, 0). That is, the X-axis and Y-axis values in the ROI of each of the reference cross section b and the third cross section a are the same and only values in the ROI of each of the reference cross section b and the third cross section a along the Z-axis depth direction is different each other, and thus it may be seen that a shape of a tissue included in the ROI indicated as the first marker 1021 may be kept constant along the Z-axis depth direction over the reference cross section b and the third cross section a.

However, in the fourth cross section image 1054, the 3D location coordinate value of the ROI may be (10, 4, 0), and the values of X-axis and Z-axis of the fourth cross section image 1054 are different from the 3D location coordinate value of the ROI of the reference cross section image 1050R. It may also be seen that the second marker 1022 displayed on the first region 1000-1 may be spaced and displayed on a different location from the first marker 1021.

FIG. 10B is an embodiment showing a case where, in an operation in which the ultrasound diagnosis apparatus successively obtains a two-dimensional (2D) cross section image, a location of the 2D cross section image in the 3D ultrasound volume data is not continuous and deviates from a predetermined range. That is, it may mean that tissues included in the ROI set in the reference cross section b are not constant along the Z-axis depth direction from the reference cross section but are located in different regions. A user may identify not only the 3D location coordinate values displayed on the second region 1000-2 of the display 1000 but also locations of the tissues included in the ROI over the plurality of cross sections through relative locations of the first marker 1021 and the second marker 1022 displayed on the first region 1000-1.

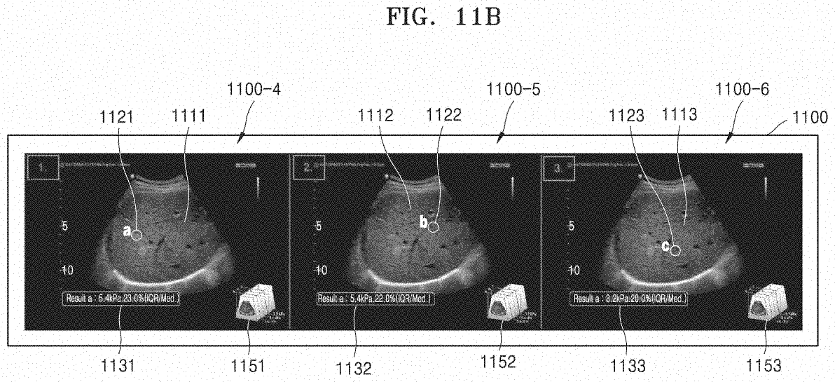

FIGS. 11A and 11B are diagrams for explaining a method, performed by an ultrasound diagnosis apparatus, of displaying thumbnail images of a plurality of cross sections of 3D ultrasound volume data together with GUIs representing shear wave data of the plurality of cross sections on images stored in a memory according to an embodiment of the disclosure.

Referring to FIG. 11A, a display 1100 of the ultrasound diagnosis apparatus may display a plurality of thumbnail images 1100-1, 1100-2, and 1100-3. The plurality of thumbnail images 1100-1, 1100-2, and 1100-3 may be images displayed by reducing the size of a plurality of 2D ultrasound images stored in the memory of the ultrasound diagnosis apparatus or a Picture Archiving and Communication System (PACS) viewer. The plurality of thumbnail images 1100-1, 1100-2 and 1100-3 may respectively include markers 1121, 1122 and 1123 indicating a ROI, first GUIs 1131, 1132, and 1133 displaying shear wave data values, and second GUIs 1141, 1142, and 1143 displaying a 3D location coordinate system.

The first GUIs 1131, 1132, and 1133 may display the shear wave data values of the ROI of the plurality of cross sections. The second GUIs 1141, 1142, and 1143 may display relative location information of each of the plurality of cross sections on the 3D location coordinate system. The second GUIs 1141, 1142, and 1143 are the same as the second GUI 620 illustrated in FIG. 6B, and redundant descriptions thereof are omitted.

Referring to FIG. 11B, the display 1100 of the ultrasound diagnosis apparatus may display a plurality of thumbnail images 1100-4, 1100-5, and 1100-6. The plurality of thumbnail images 1100-4, 1100-5, and 1100-6 may respectively include the markers 1121, 1122, and 1123 indicating the ROI, the first GUIs 1131, 1132, and 1133 displaying the shear wave data values and third GUIs 1151, 1152, and 1153.

The third GUIs 1151, 1152, and 1153 may display relative location information of each of the plurality of cross sections on a 3D volume image. The third GUIs 1151, 1152, and 1153 are the same as the first GUI 610 illustrated in FIG. 6A, and redundant descriptions thereof are omitted.

In the embodiment shown in FIGS. 11A and 11B, the ultrasound diagnosis apparatus may display GUIs displaying location information of a currently displayed cross section image, i.e., a reference cross section image, and markers indicating a location of a ROI on thumbnail images stored in the memory or the PACS viewer, and thus a user may easily identify a location of other cross section as well as a current cross section, shear wave data, and the location information of the ROI. The user may comfortably compare the relative locations of the plurality of cross sections, thereby improving user convenience.

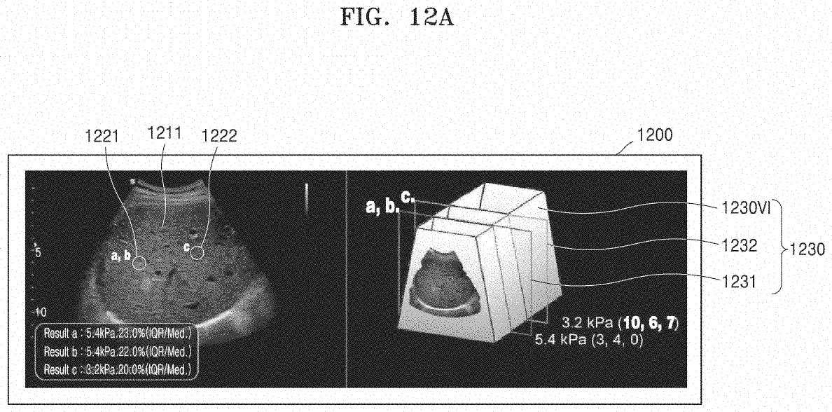

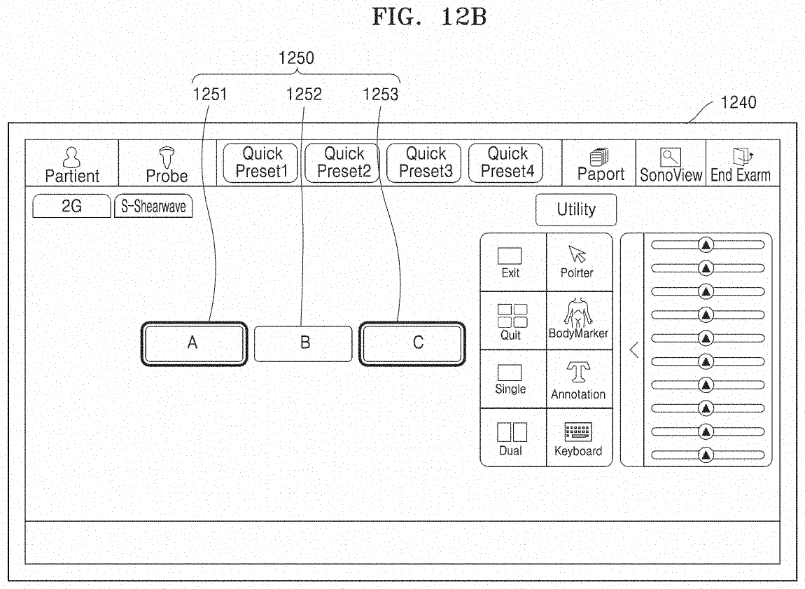

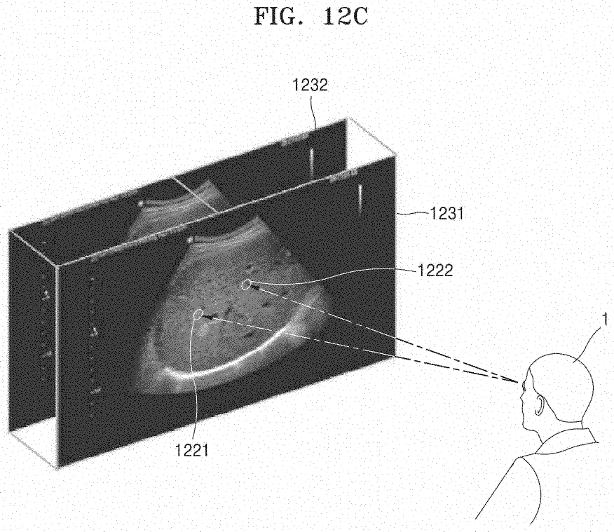

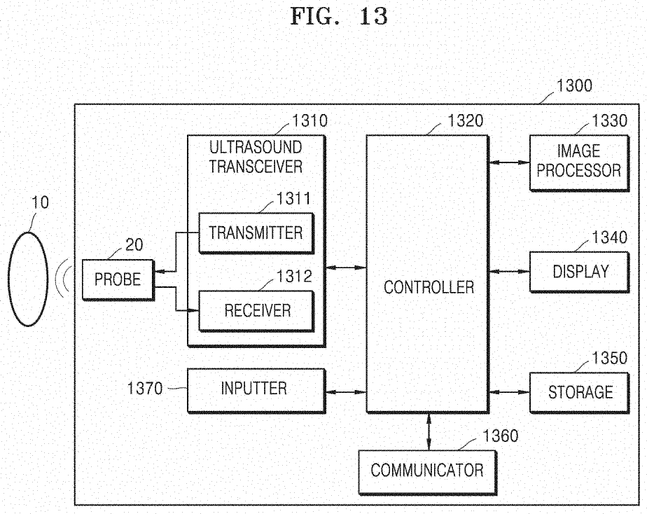

FIGS. 12A to 12C are diagrams for explaining a method performed by an ultrasound diagnosis apparatus, of displaying only at least one cross section of interest among a plurality of cross sections of 3D ultrasound volume data according to an embodiment of the disclosure.

Referring to FIG. 12A, a display 1200 of the ultrasound diagnosis apparatus may display a shear wave image 1211 of the reference cross section, markers 1221 and 1222 indicating locations of a ROI, and a GUI 1230 including a 3D volume image 1230VI. The GUI 1230 may include an image of the reference cross section b, a first cross section image 1231, and a second cross section image 1232, and may display a 3D location coordinate value of the ROI of each cross section and shear wave data. The GUI 1230 shown in FIG. 12A is the same as the first GUI 610 illustrated in FIG. 6A, and a redundant description thereof is omitted.

Locations of the markers 1221 and 1222 indicating the ROI in the shear wave image 1211 of the reference cross section may be different from each other. In an embodiment, the ROI indicated by the first marker 1221 may be the same as the reference cross section b and the first cross section a. For example, the ROI indicated by the first marker 1221 may have a 3D location coordinate value of (3, 4, 0) in the reference cross section b and the first cross section a. The ROI indicated by the second marker 1222 may have a 3D location coordinate value of (10, 6, 7) in the second cross section c. Even though the ROI is set in the shear wave image 1211 of the reference cross section, when shapes of tissues included in the ROI are not the same along a Z-axis depth direction, 3D location coordinate values of the ROI may vary depending on the plurality of cross sections.

Referring to FIG. 12B, a user input 1240 of the ultrasound diagnosis apparatus may display a GUI 1250 that receives a user input for selecting at least one of a plurality of cross sections A, B, and C. The GUI 1250 may include an A button interface 1251 receiving a user input for selecting the first cross section A, a B button interface 1252 receiving a user input for selecting the second cross section B, and a C button interface 1253 receiving a user input for selecting the third cross section C. In an embodiment, the user input 1240 may receive a user input of at least one of a click input using a mouse, a drag input using a trackball, a touch input to touch a touch screen, and combinations thereof.

In the embodiment of FIG. 12B, the user input 1240 may receive a user input that selects the A button interface 1251 and the C button interface 1252.

Referring to FIG. 12C, a user 1 may view only the selected cross sections 1231 and 1232. For example, the user 1 may easily identify a location of the ROI in each cross section through the first marker 1221 and the second marker 1222, which indicate a location of the ROI in the first cross section 1231 and the third cross section 1232 respectively.

FIG. 13 is a block diagram showing a structure of an ultrasound diagnosis apparatus 1300 according to an embodiment of the disclosure. The ultrasound diagnosis apparatus 1300 according to an embodiment may include a probe 20, an ultrasound transceiver 1310, a controller 1320, an image processor 1330, a display 1340, a storage 1350, a communicator 1360, and an inputter 1370.

The ultrasound diagnosis apparatus 1300 may be realized not only as a cart type but also as a portable type. Examples of the portable ultrasound diagnosis apparatus 1300 include, but are not limited to, a smart phone including a probe and an application, a laptop computer, a PDA, a tablet PC, and the like.

The probe 20 may include a plurality of transducers. The plurality of transducers may transmit ultrasound signals to the object 10 according to a transmission signal applied from a transmitter 1311. The plurality of transducers may receive the ultrasound signals reflected from the object 10 and form a received signal. Also, the probe 20 may be implemented integrally with the ultrasound diagnosis apparatus 1300 or may be implemented as a separate type connected to the ultrasound diagnosis apparatus 1300 by wired or wirelessly. Also, the ultrasound diagnosis apparatus 1300 may include one or a plurality of probes 20 according to an implementation.

The controller 1320 controls the transmitter 1311 to form a transmission signal to be applied to each of the plurality of transducers in consideration of locations and focusing points of the plurality of transducers included in the probe 20.

The controller 1320 performs analog-to-digital conversion of the received signal received from the probe 20, and sums the digitally converted received signal in consideration of the locations and focusing points of the plurality of transducers, to control a receiver 1312 to generate ultrasound data.

The image processor 1330 generates an ultrasound image using the ultrasound data generated by the receiver 1312.

The display 1340 may display the generated ultrasound image and various information processed by the ultrasound diagnosis apparatus 1300. The ultrasound diagnosis apparatus 1300 may include one or a plurality of displays 1340 according to an implementation. Also, the display 1340 may be implemented as a touch screen in combination with a touch panel.

The controller 1320 may control the overall operation of the ultrasound diagnosis apparatus 1300 and a signal flow between internal components of the ultrasound diagnosis apparatus 1300. The controller 1320 may include a memory storing programs or data for performing functions of the ultrasound diagnosis apparatus 1300, and a processor processing the programs or data. The controller 1320 may also receive a control signal from the inputter 1370 or an external device to control the operation of the ultrasound diagnosis apparatus 1300.

The ultrasound diagnosis apparatus 1300 includes the communicator 1360 and may be connected to an external device (for example, a server, a medical device, a portable device (smartphone, tablet PC, wearable device, etc.) through the communicator 1360.

The communicator 1360 may include one or more components that enables communication with an external device, for example, at least one of a short-range communication module, a wired communication module, and a wireless communication module.

The communicator 1360 receives a control signal and data from the external device and transmits the received control signal to the controller 1320 such that the controller 1320 controls the ultrasound diagnosis apparatus 1300 according to the received control signal.

Alternatively, the controller 1320 may transmit the control signal to the external device through the communicator 1360, thereby controlling the external device according to the control signal of the controller 1320.

For example, the external device may process data of the external device according to the control signal of the controller 1320 received through the communicator 1360.

The external device may be provided with a program for controlling the ultrasound diagnosis apparatus 1300. The program may include an instruction for performing a part or all of the operation of the controller 1320.

The program may be installed in an external device in advance, or a user of the external device may download and install the program from a server that provides an application. The server providing the application may include a recording medium storing the program.

The storage 1350 may store various data or programs for driving and controlling the ultrasound diagnosis apparatus 1300, input/output ultrasound data, and obtained ultrasound images, etc.

The inputter 1370 may receive a user input for controlling the ultrasound diagnosis apparatus 1300. For example, the user input may include an input for operating a button, a keypad, a mouse, a trackball, a jog switch, a knob, etc., an input for touching a touch pad or a touch screen, a voice input, a motion input, a bio information input (e.g., iris recognition, fingerprint recognition, etc.), and the like but is not limited thereto.

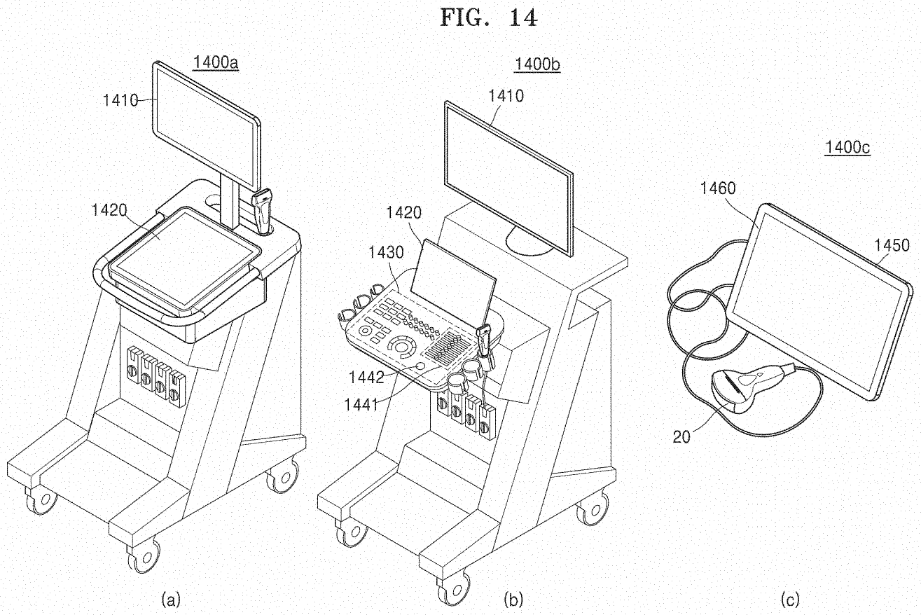

An example of the ultrasound diagnosis apparatus 1300 according to an embodiment will be described later with reference to FIG. 14.

FIG. 14 is a diagram illustrating ultrasound diagnosis apparatuses 1400a, 1400b, and 1400c according to an embodiment of the disclosure.

Referring to FIG. 14, the ultrasound diagnosis apparatuses 1400a and 1400b may include a main display 1410 and a sub display 1420. One of the main display 1410 and the sub display 1420 may be implemented as a touch screen. The main display 1410 and the sub display 1420 may display ultrasound images or various information processed by the ultrasound diagnosis apparatuses 1400a and 1400b. Also, the main display 1410 and the sub display 1420 may be implemented as a touch screen and provide a GUI, thereby receiving data for controlling the ultrasound diagnosis apparatuses 1400a and 1400b from a user. For example, the main display 1410 may display an ultrasound image, and the sub display 1420 may display a control panel for controlling displaying of the ultrasound image in the form of a GUI. The sub display 1420 may receive data for controlling displaying of an image through the control panel displayed in the form of the GUI. The ultrasound diagnosis apparatuses 1400a and 1400b may control displaying of the ultrasound images displayed on the main display 1410 using the received control data.

Referring to the ultrasound diagnosis apparatus 1400b shown in FIG. 14, the ultrasound diagnosis apparatus 1400b may further include a control panel 1430 in addition to the main display 1410 and the sub display 1420. The control panel 1430 may include a button, a trackball, a jog switch, a knob, and the like, and may receive data for controlling the ultrasound diagnosis apparatus 1400b from the user. For example, the control panel 1430 may include a Time Gain Compensation (TGC) button 1441, a Freeze button 1442, and the like. The TGC button 1441 is a button for setting a TGC value for each depth of the ultrasound image. Also, when an input of the Freeze button 1442 is detected during the scan of the ultrasound image, the ultrasound diagnosis apparatus 1400b may maintain a state in which a frame image of the point is displayed.

Meanwhile, the button, the trackball, the jog switch, the knob, and the like included in the control panel 1430 may be provided as a GUI on the main display 1410 or the sub display 1420.

Referring to the ultrasound diagnosis apparatus 1400c shown in FIG. 14, the ultrasound diagnosis apparatus 1400c may be implemented in a portable type. Examples of the portable ultrasound diagnosis apparatus 1400c include, but are not limited to, a smart phone including a probe and an application, a laptop computer, a PDA, a tablet PC, and the like.

The ultrasound diagnosis apparatus 1400c includes the probe 20 and a body 1450. The probe 20 may be connected to one side of the body 1450 by wired or wirelessly. The body 1450 may include a touch screen 1460. The touch screen 1460 may display an ultrasound image, various information processed by the ultrasound diagnosis apparatus 1400c, a GUI, and the like.

The above-described embodiments of the present disclosure may be embodied in form of a computer-readable recording medium for storing computer executable command languages and data. The command languages may be stored in form of program codes and, when executed by a processor, may perform a certain operation by generating a certain program module. Also, when executed by a processor, the command languages may perform certain operations of the disclosed embodiments.

While embodiments of the present disclosure have been particularly shown and described with reference to the accompanying drawings, it will be understood by those of ordinary skill in the art that various changes in form and details may be made therein without departing from the spirit and scope of the invention as defined by the appended claims. The disclosed embodiments should be considered in descriptive sense only and not for purposes of limitation.

1. An ultrasound diagnosis apparatus for displaying shear wave data relating to an object, the ultrasound diagnosis apparatus comprising:
a processor configured to obtain the shear wave data of the object with respect to each of a plurality of cross sections spaced apart by a predetermined distance and set a region of interest (ROI) in a reference cross section among the plurality of cross sections; and
a display displaying a graphic user interface (GUI) representing shear wave data of a region corresponding to a same location as that of the ROI in the plurality of cross sections along a depth direction.

2. The ultrasound diagnosis apparatus of claim 1, wherein the display further displays shear wave data in a region corresponding to the same location as that of the ROI in each of a first cross section spaced apart from the reference cross section in a first direction and a second cross section spaced apart from the reference cross section in a second direction opposite to the first cross section.

3. The ultrasound diagnosis apparatus of claim 1, wherein the display further displays the GUI in the form of a graph displaying distance information of the plurality of cross sections along the depth direction on an X axis and displaying the shear wave data of a region corresponding to the same location as that of the ROI in the plurality of cross sections on a Y axis.

4. The ultrasound diagnosis apparatus of claim 1, wherein the display further displays the shear wave data of the plurality of cross sections as the GUI comprising at least one of a 3D marker, a text, a dotted line, or a 3D coordinate value.

5. The ultrasound diagnosis apparatus of claim 1, wherein the display further displays a shear wave image of the reference cross section on a first region on a display screen and displays the GUI on a second region on the display screen.

6. The ultrasound diagnosis apparatus of claim 1, wherein the display further displays the GUI on a location corresponding to the ROI on a shear wave image of the reference cross section in an overlapping manner.

7. The ultrasound diagnosis apparatus of claim 1,
wherein the GUI is a three-dimensional (3D) marker that is the same as a shape of the ROI along the depth direction and displays the shear wave data of the ROI with respect to a spaced distance along the depth direction in different colors, and
wherein the display further displays the 3D marker on the ROI on an image of the reference cross section in an overlapping manner.

8. The ultrasound diagnosis apparatus of claim 1, further comprising: a user inputter configured to receive a user input for selecting a cross section image of at least two of the plurality of cross sections,
wherein the display further displays a GUI representing shear wave data of the selected cross section image of the at least two cross sections based on the user input.

9. A method of displaying shear wave data relating to an object, the method comprising:
obtaining the shear wave data of the object with respect to each of a plurality of cross sections spaced apart by a predetermined distance;
setting a ROI in a reference cross section among the plurality of cross sections; and
displaying a graphic user interface (GUI) representing shear wave data of a region corresponding to a same location as that of the ROI in the plurality of cross sections along a depth direction.

10. The method of claim 9, wherein the displaying of the GUI comprises displaying shear wave data in a region corresponding to the same location as that of the ROI in each of a first cross section spaced apart from the reference cross section in a first direction and a second cross section spaced apart from the reference cross section in a second direction opposite to the first cross section.

11. The method of claim 9, wherein the displaying of the GUI comprises displaying the GUI in the form of a graph displaying distance information of the plurality of cross sections along the depth direction on an X axis and displaying the shear wave data of a region corresponding to the same location as that of the ROI in the plurality of cross sections on a Y axis.

12. The method of claim 9, wherein the displaying of the GUI comprises displaying the shear wave data of the plurality of cross sections as the GUI comprising at least one of a 3D marker, a text, a dotted line, or a 3D coordinate value.

13. The method of claim 9, wherein the displaying of the GUI comprises displaying the GUI on a spaced location on the shear wave image of the reference cross section with the shear wave image.

14. The method of claim 9, wherein the GUI is a three-dimensional (3D) marker that is the same as a shape of the ROI along the depth direction and displays the shear wave data of the ROI with respect to a spaced distance along the depth direction in different colors, and
wherein the displaying of the GUI comprises displaying the 3D marker on the ROI on an image of the reference cross section in an overlapping manner.

15. A computer-readable recording medium having recorded thereon at least one program for performing the method of claim 1.
